(19) United States
(12) Patent Application Publication
(10) Pub. No.: US 2015/0328480 A1
(43) Pub. Date: Nov. 19, 2015

(54) Realistic Laser Acupuncture Apparatus

(71) Applicant: I-SHOU UNIVERSITY, Kaohsiung City (TW)

(72) Inventors: Chih-Yu Wang, Kaohsiung City (TW); Shu-Chen Chang, Kaohsiung City (TW); Che-Chang Kuo, Kaohsiung City (TW)

(21) Appl. No.: 14/452,566

(22) Filed: Aug. 6, 2014

(30) Foreign Application Priority Data
May 19, 2014 (TW) 103117526

(51) Int. Cl. A61N 5/06

(52) U.S. Cl. CPC A61N 5/0619; A61H 2039/005

(57) ABSTRACT

A realistic laser acupuncture apparatus includes a laser device having a laser driver and a laser head. The laser head emits a laser beam moving along a path. A focus tunable liquid lens is located in the path of the laser beam. A control unit is electrically connected to the laser device and the focus tunable liquid lens. This can substantially decrease the volume and increase the applicability of the realistic laser acupuncture apparatus.

### BACKGROUND OF THE INVENTION

1. Field of the Invention

The present invention relates to a realistic laser acupuncture apparatus and, more particularly, to a realistic laser acupuncture apparatus having a simple structure and a small volume.

2. Description of the Related Art

Acupuncture therapy originates from China and is an alternative therapy having a long history. Acupuncture includes needling and moxibustion. Needling includes inserting needles made of silver or stainless steel into acupoints of a patient to provide a therapeutic effect while the patient feels sore, pain, or numbness. A doctor can use lifting-thrusting technique or twirling technique to apply the needles into the acupoints to a required depth according to the body condition of the patient.

Traditional acupuncture is an invasive treating method. However, some patients fear needles, and there are some contraindications of needling, such as too hungry or too full. In recent years, some realistic laser acupuncture apparatuses have been proposed to proceed with non-invasive acupuncture therapy on patients. FIG. 1 shows a conventional realistic laser acupuncture apparatus 9 including a laser device 91, a convex lens 92, a concave lens 93, a polarizer 94, and a controller 95. The concave lens 92 is connected to a movable member 921. The polarizer 94 is connected to a rotating member 941. The laser device 91, the movable member 921, and the rotating member 941 are electrically connected to the control unit 95. The laser device 91 generates a laser beam moving along a path on which the convex lens 92, the concave lens 93, and the polarizer 94 are mounted in sequence. An example of such a realistic laser acupuncture apparatus 9 is disclosed in Taiwan Invention Patent No. 1398279 entitled “REALISTIC LASER ACUPUNCTURE APPARATUS IMITATING ACUPUNCTURE TECHNIQUE OF A CHINESE MEDICINE DOCTOR”.

To implement lifting-thrusting functions when using laser acupuncture, the movable member 921 is moved back and forth in a displacement distance d to change a relative spacing between the convex lens 92 and the concave lens 93, changing the focused spot of the laser beam and, hence, providing an imitative lift-thrust effect. Furthermore, the polarizer 94 can change the polarization of the light. Thus, when the control unit 95 sends a signal to the rotating member 941 to rotate the polarizer 94, the linear polarization of the laser beam can be alternated accordingly to implement the twirling effect of traditional acupuncture.

Since the realistic laser acupuncture apparatus 9 adjusts the positions of focused spot by changing the spacing between the convex lens 92 and the concave lens 93, the displacement distance d permitting movement of the convex lens 92 must be preserved, and a power unit was required in the realistic laser acupuncture apparatus 9 to move the movable member 921. Therefore, relatively large volume was necessary, which would increase the costs and limit the applicability of the realistic acupuncture laser apparatus 9 (e.g., the realistic laser acupuncture apparatus 9 is not suitable for operation in a small space). In addition, the realistic laser acupuncture apparatus 9 must include the convex lens 92 and the concave lens 93, both of which must be located in the path of the laser beam. The manufacturer of the realistic laser acupuncture apparatus 9 has to spend considerable time in adjusting the locations of the convex lens 92 and the concave lens 93 during manufacture. After-sales service for maintaining the convex lens 92 and the concave lens 93 in the predetermined locations is inevitable, which will increase the costs and time consumption.

Thus, a need exists for a realistic laser acupuncture apparatus including a simple structure to implement lift-thrust effect of acupuncture. This can substantially decrease the volume and increase the applicability of the realistic laser acupuncture apparatus. Furthermore, it is possible to reduce the time and costs of the after-sales service because the adjustment of light path between two lenses.

### SUMMARY OF THE INVENTION

An objective of an embodiment of the present invention is to provide a realistic laser acupuncture apparatus with simpler structure to eliminate the limitation and increase the applicability of the realistic laser acupuncture apparatus.

Another objective of an embodiment of the present invention is to provide a realistic laser acupuncture apparatus that the adjustment of the laser path in manufacture and alignment of lenses in after-sales in which the manufacturer of the realistic laser acupuncture apparatus does not have to spend much time and costs and can save the time and service costs of the manufacturer.

The present invention fulfills the above objectives by providing a realistic laser acupuncture apparatus which can implement the lift-thrust functions. The laser head emits a laser beam moving along a path. A focus tunable liquid lens is placed in the light path. A control unit is electrically connected to the laser device and the focus tunable liquid lens.

The realistic laser acupuncture apparatus can further include a casing having two ends. There is a space connecting both two ends. The laser head and the focus tunable liquid lens are placed in the space. The focus tunable liquid lens is located between the second end of the casing and the laser head.

The realistic laser acupuncture apparatus can further include a polarizer placed between the focus tunable liquid lens and the second end of the casing.

The realistic laser acupuncture apparatus can further include a rotating member electrically connected to the control unit. The polarizer is connected to the rotating member.

The realistic laser acupuncture apparatus can further include a collimating lens located between the laser head and the focus tunable liquid lens. The collimating lens is located in the path of the laser beam.

The second end of the casing can include adhesive materials located around the opening.

The present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 is a diagrammatic view of a conventional realistic laser acupuncture apparatus.

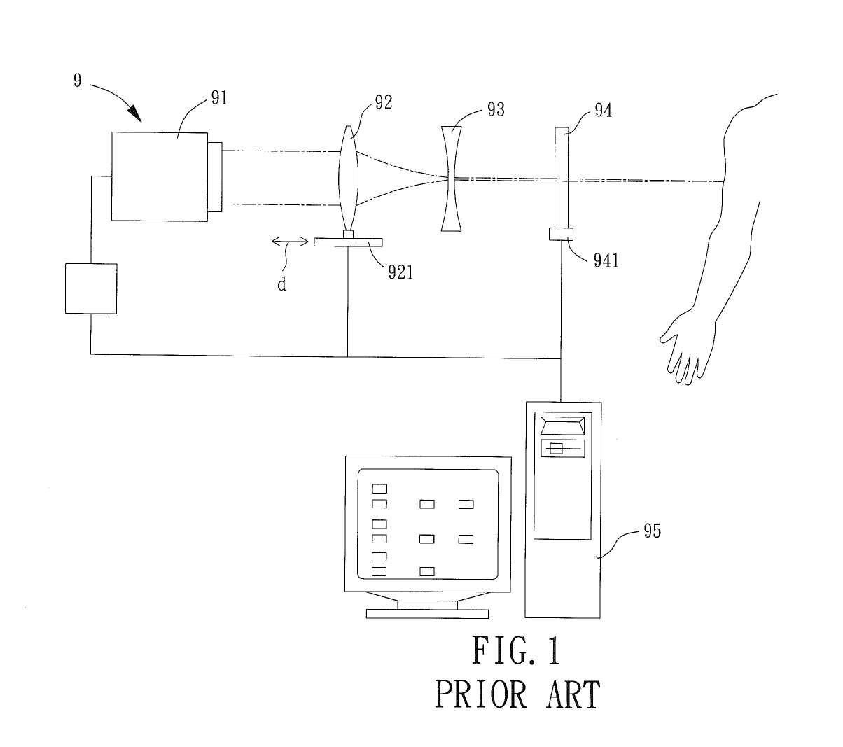

FIG. 2 is a diagrammatic view of a realistic laser acupuncture apparatus of a first embodiment according to the present invention.

FIG. 3 is a diagrammatic, partly-sectioned view of a realistic laser acupuncture apparatus of a second embodiment according to the present invention.

FIG. 4 is a schematic perspective view illustrating use of the realistic laser acupuncture apparatus of the second embodiment according to the present invention.

All figures are drawn for ease of explanation of the basic teachings of the present invention only; the extensions of the figures with respect to number, position, relationship, and dimensions of the parts to form the preferred embodiments will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

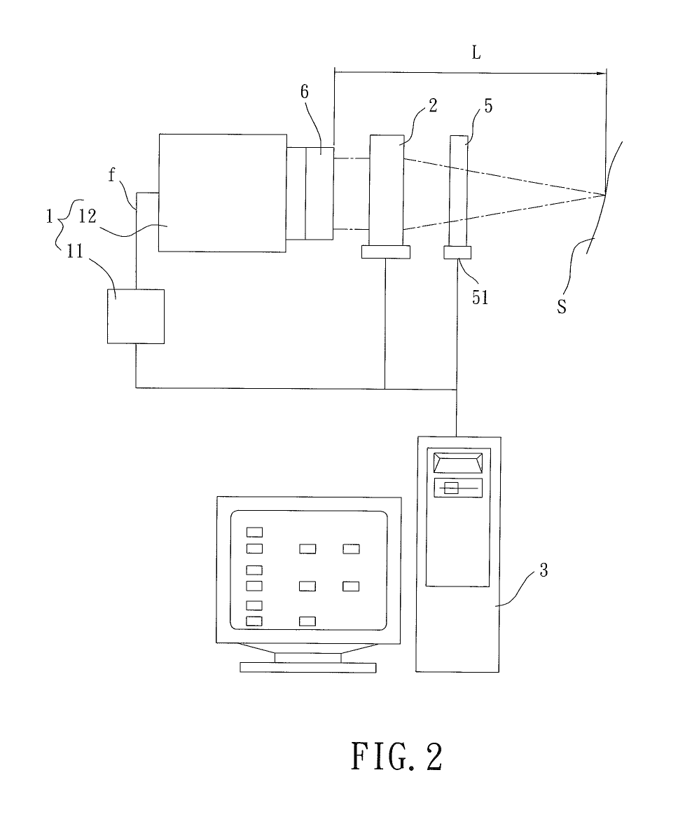

FIG. 2 is a diagrammatic view of a realistic laser acupuncture apparatus of a first embodiment according to the present invention. The realistic laser acupuncture apparatus includes a laser device 1, a focus tunable liquid lens 2, and a control unit 3. The laser device 1 generates a laser beam moving in a path L. The focus tunable liquid lens 2 is mounted in the path L of the laser beam. The control unit 3 is electrically connected to the laser device 1 and the focus tunable liquid lens 2.

The laser device 1 includes a laser driver 11 and a laser head 12 connected to the laser driver 11. Preferably, the laser driver 11 and the laser head 12 are connected by a twisted pair wire f. The laser head 12 outputs a laser beam moving along the path L to a surface S, such as a human skin. The wavelength of the laser beam is in the far infrared region (760-1000 nm). As an example, the wavelength of the laser beam can be 808 nm, because the laser beam in the far infrared region can penetrate the human skin to a deeper location without injuring the human body. The wavelength and intensity of the laser beam can be adjusted to the user needs, which can be appreciated by one having ordinary skill in the art.

The focus tunable liquid lens 2 is mounted in the path L of the laser beam and is electrically connected to the control unit 3. The focus tunable liquid lens 2 includes at least one liquid. A focus tuning effect can be provided by changing the curvature of a surface of the liquid. The focus tunable liquid lens 2 can be a hydraulic liquid lens, dielectric liquid lens, or electrowetting liquid lens. The type of the focus tunable liquid lens 2 and the type of the liquid are not limited. In a case of a hydraulic liquid lens, the focus tunable liquid lens 2 includes an optical liquid, a polymer membrane, and a circular ring. The polymer membrane envelopes the optical liquid and can be squeezed via the circular ring to change the shape of the polymer membrane.

A rapid focus tuning effect can be achieved by changing the curvature of the lens to provide different focuses. Alternatively, in a case of an electrowetting liquid lens, the focus tunable liquid lens 2 includes a chamber containing two immiscible liquids with different refractive indexes. One of the liquids is a conductive solution, and the other is a non-conductive hydrophobic liquid. A hydrophobic material is applied to a portion of an inner wall of the chamber of the focus tunable liquid lens 2 such that the conductive water solution bends towards the other portion not coated with the hydrophobic material. When electricity is supplied to the focus tunable liquid lens 2, the curvature of the surface of the conductive solution changes to alter the focal length of the focus tunable liquid lens 2. In this embodiment, the focus tunable liquid lens 2 is preferably an electrowetting liquid lens. The curvature of the liquid surface can be changed by simply adjusting the voltage of the control unit 3, and the positions of focused spot of the laser beam passing through the focus tunable liquid lens 2 can, thus, be changed.

The control unit 3 is electrically connected to the laser device 1 and the focus tunable liquid lens 2. The control unit 3 controls parameters including power on/off of the laser device 1, the intensity of the laser beam, and the wavelength. Furthermore, the curvature of the liquid surface of the focus tunable liquid lens 2 can be changed by the control unit 3 to change the lens focal length, thereby changing the focal point of the laser beam passing through the focus tunable liquid lens 2. The control unit 3 can be a circuit board or a computer. In this embodiment, the control unit 3 is a computer including a main frame and a screen. The user can control the related parameters by operating the computer with easy steps.

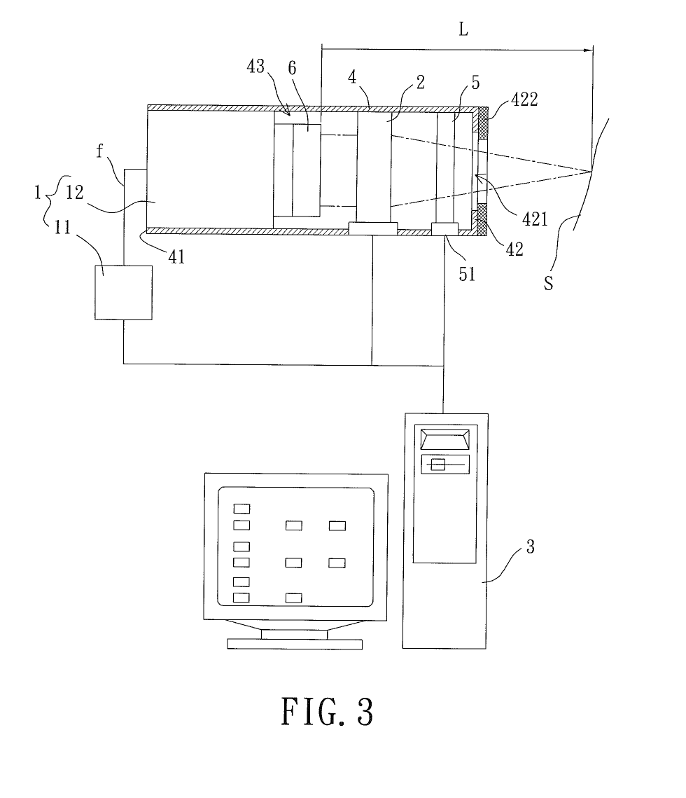

FIG. 3 is a diagrammatic, partly-sectioned view of a realistic laser acupuncture apparatus of a second embodiment according to the present invention. In this embodiment, the realistic laser acupuncture apparatus further includes a casing 4 having a first end 41 and a second end 42 opposite to the first end 41. The second end 42 of the casing 4 includes an opening 421.

The casing 4 includes a receiving space 43 in communication with the opening 421. Preferably, the second end 42 of the casing 4 includes an adhering portion 422 located around the opening 421, allowing the casing 4 to be adhered to a surface. In this embodiment, the laser head 12 and the focus tunable liquid lens 2 are received in the receiving space 43, with the focus tunable liquid lens 2 located between the second end 42 of the casing 4 and the laser head 12. Preferably, a polarizer 5 is mounted between the focus tunable liquid lens 2 and the second end 42 of the casing 4. The polarizer 5 is connected to a rotating member 51 electrically connected to the control unit 3. The control unit 3 can be controlled to drive the rotating member 51 to rotate.

Alternatively, the rotating member 51 can be manually driven to rotate. Furthermore, a collimating lens 6 is mounted in the path L of the laser beam and is located between the laser head 12 and the focus tunable liquid lens 2. Preferably, the collimating lens 6 is coupled to the laser head 12. The collimating lens 6 is used to generate collimated light, and the focus tunable liquid lens 2 focuses the laser beam to avoid energy loss due to divergence of the laser beam.

When the user is intended to use the realistic laser acupuncture apparatus according to the present invention to proceed with acupuncture, the control unit 3 is used to adjust acupuncture-related parameters including acupuncture duration or times, frequency, and intensity of lift-thrust. When the power of the laser device 1 is turned on by the control unit 3, the laser beam is outputted by the laser head 12, passes through the collimating lens 6 that keeps the laser beam from divergence. The collimated laser beam moves forward and passes through the focus tunable liquid lens 2 and illuminate on acupoints of a subject with adjustable focused spot. The focal point of the laser beam is changed to implement the lift-thrust technique of acupuncture. Specifically, by applying different voltages from the control unit 3 to the focus tunable liquid lens 2 to change the shape of the liquid in the focus tunable liquid lens 2, the focus can be changed due to a change in the curvature of the focus tunable liquid lens 2, such that the laser beam passing through the focus tunable liquid lens 2 generates different focal points at different depths of the human body to implement the lift-thrust technique of acupuncture.

Furthermore, in an embodiment including the polarizer 5, the realistic laser acupuncture apparatus according to the present invention can implement the twirling technique of acupuncture. Specifically, the rotating member 51 electrically connected to the control unit 3 can be driven by the control unit 3 to drive the polarizer 5 to rotate jointly with the rotating member 51. Alternatively, the rotating member 51 can be manually rotated. Since the laser beam passing through the polarizer 5 will generate a linear polarized light, the rotating member 51 causes the laser beam to change the polarization, thereby implementing the twirling technique of acupuncture.

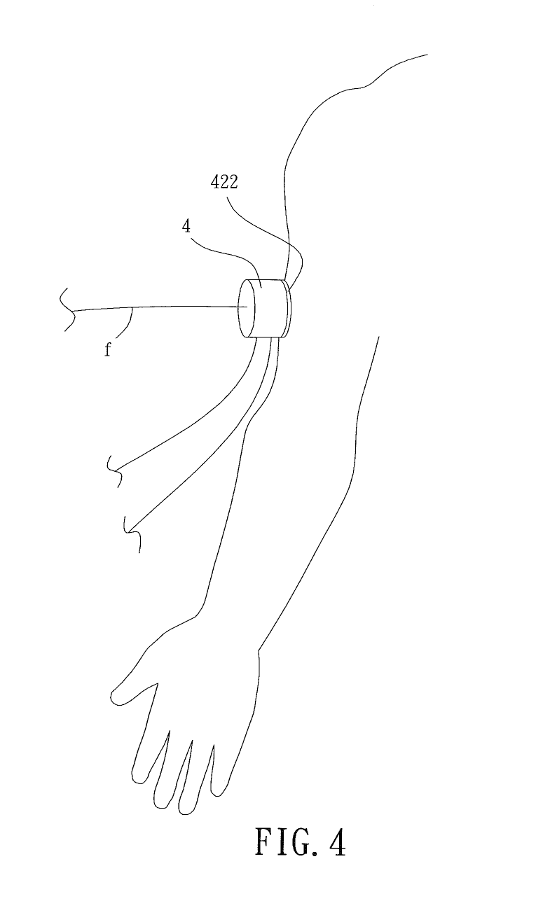

FIG. 4 is a schematic perspective view illustrating use of the realistic laser acupuncture apparatus of the second embodiment according to the present invention. Since the laser head 12 and the focus tunable liquid lens 2 are mounted in the casing 4, the laser beam outputted from the laser head 12 passes through the collimating lens 6, the focus tunable liquid lens 2, and the polarizer 5 and leaves the opening 421 to a body portion of a subject. The casing 4 can be adhered to a skin surface of the subject by the adhering portion 422. Thus, the realistic laser acupuncture apparatus can be fixed to proceed with certain manipulating methods on a specific acupoint of the body. Furthermore, provision of the casing 4 allows miniaturization of the realistic laser acupuncture apparatus such that it can be adhered to skin and thus there is no need to manually hold the realistic laser acupuncture apparatus according to the present invention, eliminating unstable factors resulting from human hand and increasing reliability of laser acupuncture.

In view of the foregoing, the realistic laser acupuncture apparatus according to the present invention includes the focus tunable liquid lens 2 electrically connected to the control unit 3 that can be operated to directly change the curvature of the focus tunable liquid lens 2 and, hence, change the focus, such that the laser beam passing through the focus tunable liquid lens 2 generates different focal points at different depths of the human body to implement the lifting and thrusting technique of acupuncture. In addition, because it is not necessary to move the focus tunable liquid lens 2, the space required for applying the realistic laser acupuncture apparatus can be substantially reduced to eliminate the application limitations. Furthermore, a manufacturer of the realistic laser acupuncture apparatus can simply adjust the relative position between the focus tunable liquid lens 2 and the laser head 12. Thus, the manufacturer of the realistic laser acupuncture apparatus does not have to spend much time and costs in adjusting the laser path or fine tuning the lens included in after-sales service, saving the time and service costs of the manufacturer.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A realistic laser acupuncture apparatus comprising:
a laser device including a laser driver and a laser head, with the laser head adapted to emit a laser beam moving along a path;
a focus tunable liquid lens located in the path of the laser beam; and
a control unit electrically connected to the laser device and the focus tunable liquid lens.

2. The realistic laser acupuncture apparatus as claimed in claim 1, further comprising: a casing including a first end and a second end, with the casing including a receiving space, with the second end of the casing including an opening in communication with the receiving space, with the laser head and the focus tunable liquid lens received in the receiving space, and with the focus tunable liquid lens located between the second end of the casing and the laser head.

3. The realistic laser acupuncture apparatus as claimed in claim 2, further comprising: a polarizer mounted between the focus tunable liquid lens and the second end of the casing.

4. The realistic laser acupuncture apparatus as claimed in claim 3, further comprising: a rotating member electrically connected to the control unit, with the polarizer connected to the rotating member.

5. The realistic laser acupuncture apparatus as claimed in claim 1, further comprising: a collimating lens located between the laser head and the focus tunable liquid lens, with the collimating lens located in the path of the laser beam.

6. The realistic laser acupuncture apparatus as claimed in claim 2, with the second end of the casing including an adhering portion, with the adhering portion located around the opening.
